(19) United States
(12) Patent Application Publication
(10) Pub. No.: US 2025/0201373 A1
(43) Pub. Date: Jun. 19, 2025

(54) Antibiotic Stewardship Program System and Method

(71) Applicant: Steve J. Williams, Basking Ridge, NJ (US)

(72) Inventor: Steve J. Williams, Basking Ridge, NJ (US)

(21) Appl. No.: 18/899,332

(22) Filed: Sep. 27, 2024

Related U.S. Application Data
(60) Provisional application No. 63/611,136, filed on Dec. 16, 2023.

(51) Int. Cl. G16H 20/10

(52) U.S. Cl. CPC G16H 20/10

(57) ABSTRACT

An antibiotic stewardship program system and method for tracking antibiotic use, in particular, antibiotics in medical facilities. The system uses human and artificial intelligence algorithms to enable the implementation of United States mandated antibiotic management, control, and reporting requirements.

### CROSS-REFERENCE TO RELATED APPLICATIONS

This application claims priority to the provisional patent application filed 16 Dec. 2023 under Ser. No. 63/611,136.

### FIELD AND BACKGROUND OF THE INVENTION

The present invention relates to an antibiotic stewardship program system for implementing and a method of use.

### BRIEF STATEMENT OF THE PRIOR ART

Certain health facilities must establish and maintain an infection prevention and control program designed to provide a safe, sanitary, and comfortable environment and to help prevent the development and transmission of communicable diseases and infections in accordance with Title 42—Public Health CHAPTER IV—CENTERS FOR MEDICARE & MEDICAID SERVICES, DEPARTMENT OF HEALTH AND HUMAN SERVICES SUBCHAPTER G—STANDARDS AND CERTIFICATION PART 483—REQUIREMENTS FOR STATES AND LONG TERM CARE FACILITIES Subpart B—Requirements for Long Term Care Facilities § 483.80 Infection control. More specifically, Section § 483.80(a) 3 mandates “An antibiotic stewardship program that includes antibiotic use protocols in the system to monitor antibiotic use.”

Antibiotic stewardship is the endeavor to measure and improve how antibiotics are prescribed by clinicians and used by patients. Such work is critical to effectively treat infections, protect patients from harms caused by unnecessary antibiotic use, and combat antibiotic resistance. The core elements include: the right drug, right dose, de-escalation to pathogen directed therapy, and right duration of therapy.

The proven benefits of antibiotic stewardship programs include optimizing antibiotic use and minimizing adverse events, such as Clostridium difficile and antibiotic resistance. The effectiveness of antibiotic stewardship programs has prompted the Centers for Disease Control and Prevention (CDC) to recommend that all hospitals have an antibiotic stewardship program.

Several guidelines were released by the CDC around the same time discussing antibiotic stewardship protocols for hospitals and how they could apply to nursing homes and what criteria would need to be used in such a setting. A significant problem was noticed in nursing homes where according to the CDC 4.1 million Americans were admitted to nursing homes during a year and up to 70% of nursing home residents receive antibiotics during a year. They also noted that up to 75% of antibiotics are prescribed incorrectly. They suggested 7 core elements for antibiotic stewardship in nursing homes which included leadership commitment, accountability, drug expertise, action, tracking, reporting, and education.

Currently, antibiotic stewardship is left to the discretion of the facility and how they wish to solve it. Most often programs list antibiotic usage on an Excel document as well as length of therapy and may include the diagnosis as an effort to track said antibiotic usage. Oftentimes this is an afterthought and done 1 to 2 months after these antibiotics have already been prescribed, thus offering no insight regarding usage or improvement of such.

The existing antibiotic stewardship programs have a number of shortcomings. In particular, most existing programs do not have a required consultation with a pharmacist or an infectious disease doctor, and are piecing together a program with their own limited knowledge base with nursing staff, nurse directors, or even administrators.

Typically, none of the people who implement the existing antibiotic stewardship programs have knowledge or expertise in prescribing pharmaceuticals and/or antibiotics. Most programs do not incorporate all elements for antibiotic stewardship that the CDC recommends and the Centers for Medicare Services (CMS) endorses. Most programs are limited in their scope and generally only offer a listing of antibiotics used and length of therapy, often on an Excel document. They do not offer solutions, protocols for antibiotic use, or monitoring of such which was the intention of the current guidance from CMS. Often this is a laborious and scattered process which can take many hours per month often done by various nursing staff or an infection preventionist who oftentimes is focused on infection prevention issues rather than antibiotic stewardship issues.

The present invention surmounts said shortcomings by incorporating all of the elements that CMS mandates and addresses all core elements of for antibiotic stewardship that the CDC recommends in their guidance including leadership commitment, accountability, drug expertise action tracking reporting and education. The present invention provides a friendly user interface for entering data on antibiotic usage allowing for that system to provide tracking of antibiotic usage and offering feedback for reporting and education. It provides drug expertise for recommending duration for antibiotics and provides the necessary accountability that his stewardship program needs. It is also fast and easy to use, taking about 30 minutes/month for data entry and compartmentalizes all findings into one convenient site for easy storage and or printing. The present invention also facilitates various local metrics to be followed allowing for ease of entry and compartmentalizing with printing or storing.

Antibiotic stewardship is important, but the ideal strategy for providing stewardship in a hospital setting is unknown. A practical, sustainable and transferable strategy is needed. The present invention discloses a system and method for antibiotic stewardship.

### SUMMARY OF INVENTION

Antibiotic data are entered by a user via a website. The data required are: prescriber, diagnosis, length of therapy, antibiotic, organism, isolation needed, various metrics, and facility or hospital-acquired. Other various metrics are known in the field, including but not limited to antibiotic use measures, clinical outcome measurements, costs, process measures, and quality measures. Traditionally, antimicrobial stewardship metrics have focused on antibiotic use, antibiotic costs, and process measures. Artificial intelligence analyzes the data in order to recommend a diagnosis and duration for therapy.

### BRIEF DESCRIPTION OF DRAWINGS

FIG. 1 is a schematic of the system elements and specification methods.

FIG. 2 is a flowchart illustrating the logic of the invention.

### DETAILED DESCRIPTION OF THE INVENTION

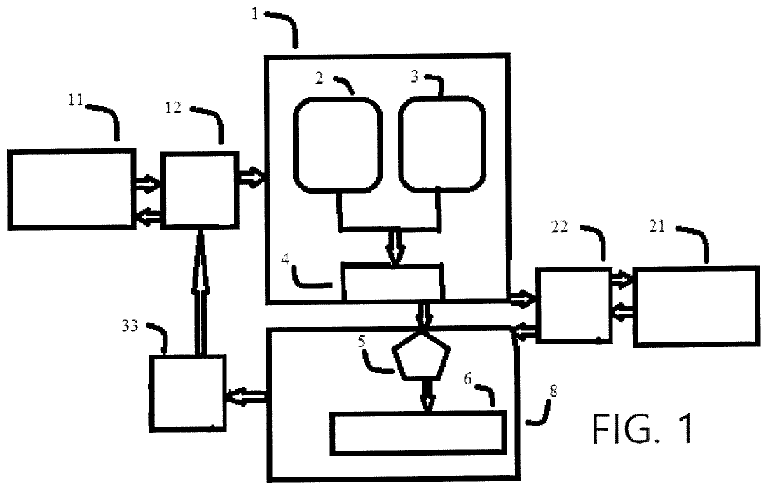

Disclosed is a system adapted to run an antibiotic stewardship program. Referring now to FIG. 1, said system comprises a data server 1 comprising medical data 2, health actions 3, and an indexing function 4. Said indexing function 4 integrates medical data 2 and health actions 3 so as to allow proper application as to a patient for which a user is employing the invention. Also provided is an advice computer 8, comprising an artificial intelligence (AI) algorithm-generator 5, and memory 6.

When user 11 initiates a query using first communications device 12, communicating patient information to data server 1 which segregates elements of patient information into medical data 2 and health actions 3 storage areas. When segregation and recording is completed, data server 1 signals program 4 which selects appropriate data to be served to either human algorithm or Artificial Intelligence (AI) algorithm, wherein human algorithm is the output of a person such a medical doctor or healthcare professional who uses professionally certified training to recommend specific course(s) of action such as drug identification or dosage, without limitation. If human algorithm then same is communicated by second communication device 22; if AI algorithm, then same is communicated to the advice computer 8.

Said data server 1 transmits data to advice computer 8 and second communication device 22. Users 11 are in two-way communication through a first communication device 12 which communicates with said data server 1 and receives data from a third communication device 33. Said third communication device 33 also receives data from advice computer 8 which in turn receives data from second communication device 22. The system further provides for a human algorithm site 21 which may be either a machine or a medical specialist, which human algorithm site 21 is in two-way communication with a second communication device 22.

A user enters antibiotic data for a specific antibiotic, prescriber, diagnosis, length of therapy, organism, isolation needed, various metrics, and facility or hospital-acquired.

Once the data have been submitted, the data are analyzed by algorithms in order to validate and prepare a recommendation regarding diagnosis and length of therapy. There are two primary embodiments of the invention: one using human algorithm, another using artificial intelligence (or “AI”) algorithm.

### Using Human Algorithm

More particularly, the algorithm proceeds as follows:

Systemically, referring now to FIG. 1, user 11 communicates to data server 1 via a first communication device 12.

Data server 1 records patient information in medical data 2 and health actions 3.

Data server signals indexing 4 when said patient information is recorded.

Indexing 4 formats patient information from medical data 2 and health actions 3,

Data server 1 signals human algorithm site 21 via a second communication device 22 if human algorithm is invoked.

Human algorithm site 21 formulates recommendation(s) and communicates said recommendation(s) to advice computer 8.

Advice computer 8 records said recommendation(s) and communicates said recommendation(s) via a third communication device 33 to said first communication device 12.

Said User 11 receives said recommendation via first communication device 12.

Additionally, further antibiotic oversight can be employed if a facility has a provider that oversees antibiotic usage, or a steward. The World Health Organization's 2000 report on world health broadly defined stewardship as “the careful and responsible management of the well-being of the population”. This person can receive information regarding antibiotic usage from the facility that has already gone through analysis by present invention's algorithms and make even further recommendations through an app acquired on the Apple App Store through IOS or through Google Play for Android, wherein the present invention's algorithms are either traditional software algorithms or AI-generated algorithms.

### Using AI Algorithm

User 11 communicates to data server 1 via first communication device 12.

Data server 1 records patient information in medical data 2 and health actions 3.

Data server 1 signals Indexing 4 when said patient information is recorded.

Indexing 4 formats patient information from medical data 2 and health actions 3.

Data server 1 signals advice computer 8 if artificial intelligence algorithm generator 5 is invoked.

Artificial intelligence algorithm generator 5 recommendation and communicates to advice computer 8.

Advice computer 8 records said recommendation and communicates said recommendation via third communication device 33 to said first communication device 12.

Said User 11 receives said recommendation via first communication device 12. Communication between elements is conducted via a communications network 7 (not shown),

Alternatively, antibiotic data can also be entered through an app obtained on the App Store through IOS or Google play for android. The data required are prescriber, diagnosis, length of therapy, antibiotic, organism, isolation needed, various metrics, and facility or hospital-acquired.

Once the data are submitted, the present invention's algorithms analyze the data, then display a recommendation regarding diagnosis and length of therapy. The algorithm to do so is the same as noted above.

### Preferred AI Embodiment

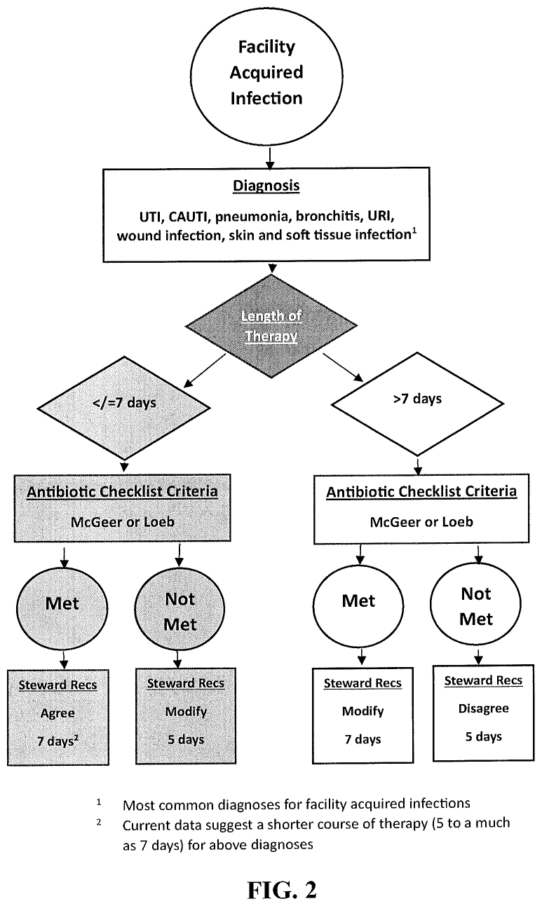

The preferred embodiment of the AI element per se for the present invention takes the form of an electronic programmable read only memory (EPROM) which could be programmed considering the patients information and guidelines The EPROM assists in “PARSING” the presentations to at least one healthcare provider which will be triggered/selected by the healthcare provider's response to a previous question. Examples of the “parsing” used in the system are given hereinafter in conjunction with the explanation of FIG. 2.

Now referring to FIG. 2. the Healthcare provider when using the present invention for a Facility Acquired Infection query, will enter the diagnosis (see top box on FIG. 2.—“uti, cauti, pneumonia . . . ”) the algorithm will ask length of therapy and the algorithm will parse the presentation to the healthcare provider based on the answer to the question length of therapy (i.e., “</=7 days” or “>7 days”). The algorithm will proceed to the “Antibiotic Checklist Criteria” (i.e., “Met” or “Not Met”) and so on.

### Doctor's Response System

In order for the disclosed system to properly function, patient information prescriber, diagnosis, length of therapy, antibiotic, organism, isolation needed, various metrics, and health care facility are recorded for use by a doctor with the application of the disclosed artificial intelligence and executing a response during an antibiotic stewardship monitoring encounter with a healthcare provider to be monitored.

For example, the doctor's response may be activated when the communication device requests the monitoring system to start upon receiving a signal from said communication device.

The Doctor system is initiated by appropriate questions from the healthcare provider, the doctor recognizes (i.e., “listens for”) the healthcare provider's answers, updates the healthcare provider's database, directs the healthcare provider monitoring interaction, and advises the healthcare provider. The information acquired from the healthcare provider call is available to medical practitioners on both a real-time basis when the calls are being made, or on an ad-hoc basis after the calls are logged.

In the preferred embodiment upon receiving a call from the healthcare provider the advice computer 8 would generate and transmit at least one question which the advice computer 8 would select from a choice of typical questions. Depending on and responsive to the advice computer's 8 answers, the advice computer 8 might respond with one of the following responses: “change length”, “change diagnosis”, or “validate response” or “invalidate response”. The encounter could continue through some more questions, each time the advice computer 8 parsing to match an unfilled portion of the question from several choices, based on the advice computer's 8 institutional knowledge and the healthcare provider's response.

### Artificial Intelligence (AI) and Parsing Response System

In order for the disclosed system to properly function, patient information prescriber, diagnosis, length of therapy, antibiotic, organism, isolation needed, various metrics, and health care facility are recorded for use with the application of the disclosed artificial intelligence and parsing during an antibiotic stewardship monitoring encounter with a healthcare provider to be monitored.

For example, the automatic monitoring (note, the healthcare provider is not required to activate the automatic monitoring device) may be activated when the artificial intelligence system commands the voice system to request the monitor to start upon receiving a signal said communication device has been activated, and the recording function takes place.

The automatic monitoring device might use AT&T's Dual-Tone Matrix Frequency (DTMF) standard for communication devices such as touch-tone telephones, which DECvoice hardware (the Voice Synthesis/Recognition technology) can recognize. An encoding device is used to translate the analog signals coming from said monitoring instruments to the telephone.

The Artificial Intelligence system is driven by a simple to use Natural Language interface which directs the Voice system to send (“speak”) appropriate questions, recognize (“listen for”) the healthcare provider's answers, update the healthcare provider's database, direct the healthcare provider monitoring the interaction, and advise the healthcare provider. The information acquired from the healthcare provider call is available to medical practitioners on both a real-time basis when the calls are being made, or on an ad-hoc basis after the calls are logged.

In the preferred embodiment upon receiving a call from the healthcare provider the artificial intelligence would generate and transmit at least one question which the artificial intelligence program would select from a choice typically questions. Depending on and responsive to the artificial intelligence's answers, the artificial intelligence program will parse, i.e., match question with an appropriate selection. When asked typical questions, then the artificial intelligence might respond with one of the following: “change length”, “change diagnosis”, “validate response” or “invalidate response”.

The encounter could continue through some more questions, each time the artificial intelligence program parsing to match an unfilled portion of the question from several choices, based on the artificial intelligence program and the healthcare provider's response.

The present invention may be combined with artificial intelligence software. Such a combination would result in the transformation of entry information, mining antibiotic options to facilitate lower drug cost; optimize drug combination; ameliorate medical difficulties associated with adverse drug interactions and allergies. Additionally, combining artificial intelligence with the present invention will allow point click and plan of care others with electronic health record (EHR) systems such as EPIC in the future.

The AI addition is confirmed to leverage Agentic AI and other applications to simplify the process of supervised assistive stewardship process. The applications will extend to other areas where secondary advice can be shared e.g. international expert consultative process, Or creating a cohort of experts to provide advice, stewardship for care beyond infectious diseases.

Agentic AI applications will be to streamline the workflow processes. E.g. sharing the data from and to the medical records via autonomous entry using voice, free form text etc. This can be supervised.
a. Limited memory & Reinforcement Learning AI will assist in the process of algorithmic decision making in diagnostics and care. The AI component will be to autonomously detect patterns and advise on the likely algorithms.
b. Reactive AI to create the proactive alerts based on the diagnostic data e.g. Sepsis
c. Generative AI to create reports and dashboards for IDSteward's native data or data ingested from the EHR systems such as PCC, or EPIC.

The addition of AI will significantly leverage the additional data from EHR systems becomes available.

As with other applications AI will assist in the process by eliminating the human error. Additionally, AI will take advantage of other nonlinear medical technology to improve the quality of care as stewardship. It will also support the quality of care and stewardship by providing access to the experts without physical presence constraints.

An example question which will be asked to the AI with an example AI answer; and for certain IDs AI can find the patterns in diagnostic or patients' behavior. E.g. what other diagnosis have similar symptoms that we did not connect earlier. Is there a pattern in patients' symptoms or diagnostics that we are not seeing due to limited data set availability.

What are the best results? How can we optimize on the prescriptions? The AI will be trained on native data as well as the EHR data available to the system. We will take standard (government recommended and approved) care to enhance governance and ethical considerations using our reusable automation framework for testing (RAFT) framework. Reliable and secure, Accountable and governed, Fair and human centered, transparent and explainable.

More specifically, the present invention may be combined with computer-based segmentation of medical word data, and more particularly to computer-based artificial intelligence-based segmentation of target word structures in medical data.

Medical word data segmentation is an important technology that supports the entire clinical imaging workflow from diagnosis, patient stratification, therapy planning, intervention, and follow-up. Medical word data segmentation refers to the detection of boundaries of word structures, such as patients name, organs, vessels, different types of tissue, pathologies, medical devices, etc., in medical records of a patient. Automatic segmentation of anatomical objects is a prerequisite for many medical record analysis tasks, such as patent drug tracking, disease diagnosis, and quantification. Medical record registration is used in a large number of applications to detect various changes in patient condition and status. Due to the vast range of applications to which medical word data segmentation can be applied, it is challenging to develop a general medical word data segmentation method that works robustly for all uses.

The present invention provides methods and systems for artificial intelligence-based segmentation of medical word data. Embodiments of the present invention provide multiple artificial intelligence based medical word data segmentation methods, including multiple different deep learning based medical word data segmentation methods. Embodiments of the present invention also provide a method and system for autonomous artificial intelligence based medical word data segmentation in which a trained intelligent artificial agent performs intelligent automated recognition of segmentation tasks and intelligent automated selection and application of segmentation algorithms. This allows the intelligent artificial agent to be applied to intelligently perform various different segmentation tasks, including segmentation of different anatomical structures and segmentation in different medical imaging modalities.

In one embodiment of the present invention, a medical record of a patient is received. A current segmentation context is automatically determined based on the medical record and at least one segmentation algorithm is automatically selected from a plurality of segmentation algorithms based on the current segmentation context. A target anatomical structure is segmented in the medical record using the selected at least one segmentation algorithm.

The present invention includes a system for intelligent autonomous medical word data segmentation according to an embodiment of the present invention; a method for intelligent autonomous medical word data segmentation according to an embodiment of the present invention; a method for training a deep learning architecture for anatomical object segmentation using a joint learning framework to integrate priors according to an embodiment of the present invention; a joint training framework for training a DNN architecture according to an embodiment of the present invention; a method of segmenting a target medical word structure using a deep neural network with integrated priors according to an embodiment of the present invention; a method for deep reinforcement learning (DRL) based segmentation of a non-rigid anatomical object in a medical record according to an embodiment of the present invention.

The present invention relates to artificial intelligence-based segmentation in medical records. Embodiments of the present invention are described herein to give an understanding of the medical word data segmentation methods. A digital record is often composed of digital representations of one or more letters (or numbers). The digital representation of an object is often described herein in terms of identifying and manipulating the objects. Such manipulations are virtual manipulations accomplished in the memory or other circuitry/hardware of a computer system. Accordingly, is to be understood that embodiments of the present invention may be performed within a computer system using data stored within the computer system or available through a network system.

Medical word data segmentation refers to the detection of boundaries of word structures, such as patients name, organs, vessels, different types of tissue, pathologies, medical devices, etc., in medical records of a patient. Embodiments of the present invention provide multiple artificial intelligence based medical word data segmentation methods, including multiple different deep learning based medical word data segmentation methods. Embodiments of the present invention also provide a method and system for autonomous artificial intelligence based medical word data segmentation in which a trained intelligent artificial agent performs intelligent automated recognition of segmentation tasks and intelligent automated selection and application of segmentation algorithms.

In an advantageous embodiment of the present invention, a method and system for autonomous artificial intelligence based medical word data segmentation utilize a trained intelligent artificial agent to perform intelligent automated recognition of segmentation tasks and intelligent automated selection and application of segmentation algorithms. This allows the intelligent artificial agent to be applied to intelligently perform various different segmentation tasks, including segmentation of different anatomical structures and segmentation in different medical imaging modalities. The intelligent artificial agent can intelligently select one or a combination of segmentation algorithms from a plurality of segmentation algorithms to perform medical word data segmentation for various medical record retrievals. Accordingly, instead of a user having to select an appropriate segmentation technique to perform a particular segmentation task, the artificial intelligent agent can be used to intelligently and autonomously select and apply an optimal segmentation algorithm or combination of segmentation algorithms for any segmentation task.

A system for intelligent autonomous medical word data segmentation according to an embodiment of the present invention starts with a master segmentation artificial agent is run on a computer system. The computer system communicates with one or more word data acquisition device, a word or number archiving and communication system (WACS), and a segmentation algorithm database. The computer system can be implemented using any type of computer device and includes computer processors, memory units, storage devices, computer software, and other computer components. In one embodiment, the computer system can be implemented using a local computer device with respect to the word data acquisition device and/or the WACS. In a possible implementation, the computer system running the master segmentation artificial agent and the word data acquisition device can be implemented as a single device. In another possible implementation, the computer system running the master segmentation artificial agent can be implemented as part of the WACS. In another possible implementation, the computer system running the master segmentation artificial agent can be implemented or as a separate local computer device (e.g., workstation) that communicates wirelessly or via a direct wired connection with the word data acquisition device and/or the WACS. In a possible embodiment, the computer system running the master segmentation artificial agent can be a mobile device, such as a smart phone or tablet. In another possible embodiment, the computer system running the master segmentation artificial agent can be implemented on a remote cloud-based computer system using one or more networked computer devices on the cloud-based computer system. In this case, medical word data of patients can be transmitted to a server of the cloud-based computer system, the master segmentation artificial agent can be run as part of a cloud-based service to perform medical record registration, and the segmentation results can then be returned to a local computer device.

The word data acquisition device can be any type of medical word data acquisition device, such as a coper scanner data can be sent to the computer system running the master segmentation artificial agent and/or stored in the WACS. Multiple word data acquisition devices may communicate with the computer system running the master segmentation artificial agent. The WACS stores medical word data of various record scanning systems for various patients in a digital format. For example, the WACS can use the Digital Imaging and Communications in Medicine (DICOM) format for storage and transfer of medical records. The computer system running the master segmentation artificial agent can retrieve medical word data stored in the WACS. Segmentation results extracted from the medical word data can also be stored in the WACS.

The segmentation algorithm database stores a plurality of automated artificial intelligence-based segmentation algorithms. Each segmentation algorithm stored in the segmentation algorithm database includes a set of computer program instructions that define a computer-based method for automatic medical word date segmentation. When the master segmentation artificial agent one or more of the segmentation algorithms stored in the segmentation algorithm database to perform a medical word data segmentation task, the corresponding computer program instructions can be loaded into a memory of the computer system can run on one or more processors of the computer system to perform the segmentation task. In a possible implementation, the segmentation algorithm database can be stored in a storage device of the computer system running the master segmentation artificial agent. In another possible implementation, the computer system running the master segmentation artificial agent can access the segmentation algorithm database via a local network. In another possible implementation, the segmentation algorithm database can be stored in a cloud-based computer system, and the computer system running the master segmentation artificial agent can access the segmentation algorithm database via a remote server over a data network, such as the Internet.

The segmentation algorithms stored in the segmentation algorithm database can include a plurality of deep learning based medical word data segmentation methods, each of which including a respective trained deep neural network architecture for performing medical word date segmentation. For example, the segmentation algorithms can include the deep learning based segmentation algorithms described below, including segmentation using a deep neural network (DNN) that integrates shape priors through joint training, non-rigid shape segmentation method using deep reinforcement learning, segmentation using deep learning based partial inference modeling under domain shift, segmentation using a deep-medical word-to-medical word network and multi-scale probability maps using a recurrent neural network (RNN). The segmentation algorithm database may include other deep learning-based segmentation algorithms as well, such as marginal space deep learning (MSDL) and marginal space deep regression (MSDR) segmentation.

The segmentation algorithm database stores multiple versions of each segmentation algorithm corresponding to different target anatomical structures and different medical imaging modalities. For deep learning-based segmentation algorithms, each version corresponding to a specific target word data structure. Accordingly, when the master segmentation artificial agent selects one or more segmentation algorithms from the those stored in the segmentation algorithm database, the master segmentation artificial agent selects not only the type of segmentation algorithm to apply, but the specific versions of segmentation algorithms that are best for performing the current segmentation task.

The master segmentation artificial agent is a trained intelligent artificial agent that automatically recognizes a current segmentation context based on medical word data of a patient and automatically selects one or more of the segmentation algorithms in segmentation algorithm database to perform segmentation of the medical word databased on the current segmentation context. The master segmentation artificial agent is an intelligent artificial agent that is implemented on one or more computers or processors of computer system by executing computer program instructions (code) loaded into memory. The master segmentation artificial agent observes the medical word date to be segmented and autonomously acts to select a segmentation strategy using a segmentation policy learned using machine learning.

According to an advantageous embodiment, the master segmentation artificial agent can select an optimal segmentation strategy for different word record types. A pre-trained segmentation algorithm that has not been trained on a large database of such new medical word may not have the ability to generalize on this new data. The master segmentation artificial agent can automatically manage and orchestrate a set of segmentation algorithms to achieve a desired segmentation task. For example, the master segmentation artificial agent may first analyze the medical word data to be segmented and, based on the analysis of the medical data record, determine versions of one or more of the segmentation algorithms with parameter settings that will achieve the best segmentation results for the target segmentation task. The master segmentation artificial agent may select a single segmentation algorithm (version) to perform the segmentation or may select multiple segmentation algorithms and then fuse the segmentation results from the selected segmentation algorithms and output a unified segment result.

The master segmentation artificial agent can also perform online adaptation of the segmentation algorithms. For example, the master segmentation artificial agent can control one or more of the segmentation algorithms in the segmentation algorithm database to be re-trained based on new training data. In a possible embodiment, one or more of the segmentation algorithms stored in the segmentation algorithm database can be deep learning segmentation algorithms with respective trained deep neural networks that were acquired pre-trained or trained using publicly available data, and the master segmentation artificial agent can control those segmentation algorithms to be re-trained using medial word data of domain specific to a clinical site at which the master segmentation artificial agent is running or using medical word data that is private to the clinical site. In this way the master segmentation artificial agent can more specifically tailor the trained deep learning segmentation algorithms available in the segmentation algorithm database to the specific segmentation tasks performed at the clinical location without transmitting private patient data to an outside party for training.

The master segmentation artificial agent can be trained based on training data including medical word data and known ground truth segmentation results for given segmentation tasks. Segmentation can be performed on each of the training samples using each of the segmentation algorithms stored in the segmentation algorithm database and the resulting segmentation results can be compared to the ground truth segmentation results to calculate confidence measures for each of segmentation algorithms. Synthetic training samples can also be generated from the real medical word data training samples by converting the real medical word data training samples to synthetic medical word data having different word data record characteristics (e.g., hospital record, doctor record, drug use record etc.). For example, a deep neural network (DNN) can be trained to deep learning techniques, such as deep reinforcement learning, to select one or more segmentation algorithms for a given segmentation task based on characteristics of the medical word data to be segmented. At runtime, when a medical word data to be segmented is received, the master segmentation artificial agent uses the trained machine learning based mapping to select the best segmentation algorithm or combination of segmentation algorithms to perform the segmentation task based on the medial word characteristics of the received medical word data. In an exemplary implementation in which the master segmentation artificial agent uses a trained DNN to select the one or more segmentation algorithms, the medical word data can be directly input to the trained DNN, which can automatically extract characteristics or features used to determine which segmentation algorithm or algorithms to select.

In another advantageous embodiment, the master segmentation artificial agent can be applied to select an optimal segmentation strategy across multiple different medical records. Typically, medical word data segmentation algorithms are designed and optimized with a specific context of use. For example, algorithms designed for segmenting date structures generally perform well in United States data formats. The master segmentation artificial agent can automatically identify the context of use European date formats (e.g., the target anatomical structure to be segmented) and automatically switch between different segmentation algorithms for different targeted and structures.

A machine learning based classifier (e.g., probabilistic boosting tree (PBT), random forests classifier, deep neural network (DNN), etc.) can be trained to recognize a word data entity in a view of a medical record. In a possible implementation, as a user visualizes a medical record on a screen, the trained classifier can be applied to automatically detect what word data or record is currently being visualized on the screen. The master segmentation artificial agent can then select one or more segmentation algorithms for segmenting the related records.

Although the master segmentation artificial agent acts autonomously to select one or more segmentation algorithms, in a possible implementation, a user (or a clinical site) may be provided with a manual override option (for example on a user interface displayed on a display device) that allows the user to override the master segmentation artificial agent and manually chose a specific segmentation algorithm. Rules controlling the use of the manually override can be defined and/or adjusted by a user.

Other modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein but are to be accorded the widest scope consistent with this disclosure, the principles and the novel features disclosed herein.

### REFERENCE SIGNS LIST

1. Data server
2. Medical data
3. Health actions
4. Indexing function
5. Artificial intelligence algorithm generator
6. Memory
7. Communications network (not shown)
8. Advice computer
9-10 [reserved]
11. Users
12. First communication device
13-20 [reserved]
21. Human algorithm site (may be machine or medical specialist)
22. Second communication device
23-32 [reserved]
33. Third communication device

What is claimed is:

1. A system for implementing an antibiotic stewardship program for users, comprising:
a data server,
medical data,
health actions,
an indexing function,
wherein said indexing function integrates said medical data and said health actions for proper application as to a patient;
an artificial intelligence algorithm generator,
memory,
a communications network,
a human algorithm site,
an advice computer,
a first communication device,
a second communication device, and
a third communication device.

2. The system of claim 1, wherein said human algorithm site is a medical specialist.

3. The system of claim 1, wherein said human algorithm site is a machine.

4. The system of claim 1, wherein said memory comprises an EPROM.

5. A method for implementing an antibiotic stewardship program for monitoring of at least one patient, comprising the steps of:
a) providing the system of claim 1,
b) using a communication device,
c) using an advice computer through a computer monitoring device,
wherein said computer monitoring device is used by at least one healthcare provider without requiring said at least one healthcare provider to activate said computer monitoring device;
d) recording patient information, wherein said patient information comprises a prescriber, a diagnosis, a length of therapy, an antibiotic, an organism, an isolation needed indicator, at least one defined metric, and a healthcare facility;
e) creating an antibiotic stewardship monitoring encounter with said at least one healthcare provider to be monitored by said doctor;
f) said advice computer parsing at least one question to create a selection of questions and a sequence of said selection of questions chosen by said advice computer from said selection of questions based on progressive responses from said at least one healthcare provider;
g) obtaining a signal from said monitoring device through said computer monitoring device at the end of said sequence; and
h) applying criteria created by said doctor for evaluating said at least one healthcare provider's response and an obtained reading signal to display a recommendation regarding said diagnosis and said length of therapy.

6. A system and method of remote antibiotic stewardship monitoring of at least one patient by using a communication device and artificial intelligence (AI) algorithms through a computer monitoring device which is used by at least one healthcare provider without requiring said at least one healthcare to activate said computer monitoring device, comprising the steps of:
a) recording patient information including a prescriber, a diagnosis, a length of therapy, an antibiotic, an organism, an isolation needed indicator, at least one metric, and a health care facility;
b) generating at least one question which could be asked of said AI algorithms by said at least one healthcare provider, said at least one question being chosen by said at least one healthcare provider;
c) creating an antibiotic stewardship monitoring encounter with said at least one healthcare provider to be monitored by said AI algorithms;
d) using said AI algorithms for parsing said at least one question to create a selection and a sequence of selected questions chosen by said AI algorithms based on progressive responses from said at least one healthcare provider;
e) obtaining a signal from said monitoring device through said communication device at the end of said sequence of said selected questions, and
f) applying said criteria created by said AI healthcare provider for evaluating said at least one healthcare provider's response and an obtained reading signal to display recommendations regarding said diagnosis and said length of therapy necessary.
